SEQUENCE LISTING (3 sequences)

SEQ ID NO: 1 - protein, 151 aa, Homo sapiens
MRFLAATFLLLALSTAAQAEPVQFKDCGSVDGVIKEVNVSPCPTQPCQLSKGQSYSVNVTFTSNIQSKSSKAVVHGILMGVPVPFPIPEPDGCKSGINCPIQKDKTYSYLNKLPVKSEYPSIKLVVEWQLQDDKNQSLFCWEIPVQIVSHL

SEQ ID NO: 2 - protein, 149 aa, unidentified
MRFLAATILLLALVAASQAEPLHFKDCGSKVGVIKEVNVSPCPTDPCQLHKGQSYSVNITFTSGTQSQNSTALVHGILEGIRVPFPIPEPDGCKSGINCPIQKDKVYSYLNKLPVKNEYPSIKLVVEWKLEDDKKNNLFCWEIPVQITS

SEQ ID NO: 3 - protein, 16 aa, synthetic construct
EFPKPSTPPGSSGGAP

(19) United States
(12) Patent Application Publication
(10) Pub. No.: US 2026/0102463 A1
(43) Pub. Date: Apr. 16, 2026

(54) ENGINEERING NON-CYTOTOXIC DELIVERY OF PROTEINS BY T CELLS VIA FUSION TO NPC2

(71) Applicant: UNIVERSITY OF HOUSTON SYSTEM, Houston, TX (US)

(72) Inventors: Navin Varadarajan, Houston, TX (US); Arash Saeedi, Houston, TX (US)

(73) Assignee: UNIVERSITY OF HOUSTON SYSTEM, Houston, TX (US)

(21) Appl. No.: 19/114,034

(22) PCT Filed: Sep. 21, 2023
(86) PCT No.: PCT/US2023/033348

§ 371 Date: Mar. 21, 2025

Related U.S. Application Data
(60) Provisional application No. 63/408,729, filed on Sep. 21, 2022.

(51) Int. Cl. A61K 38/17; A61K 40/11; A61K 40/15; A61K 40/31; A61P 35/00

(52) U.S. Cl. CPC A61K 38/1709; A61K 40/11; A61K 40/15; A61K 40/31; A61P 35/00; A61K 2239/22

(57) ABSTRACT

Embodiments of the present disclosure pertain to modified cells that include a fusion protein of NPC intracellular cholesterol transporter 2 protein (NPC2) and another protein. Additional embodiments of the present disclosure pertain to methods of delivering a protein to a target cell by associating the target cell with a modified cell of the present disclosure, which includes a fusion protein of NPC2 and the protein to be delivered. Further embodiments of the present disclosure pertain to methods of treating or preventing a condition in a subject by administering to the subject a modified cell of the present disclosure. Additional embodiments of the present disclosure pertain to methods of making the modified cells of the present disclosure by introducing a fusion protein of the present disclosure to cells.

### CROSS-REFERENCE TO RELATED APPLICATIONS

This application claims priority to U.S. Provisional Patent Application No. 63/408,729, filed on Sep. 21, 2022. The entirety of the aforementioned application is incorporated herein by reference.

### STATEMENT UNDER 37 C.F.R. § 1.34(C)(1)

Pursuant to 37 C.F.R. § 1.834, Applicant hereby submits a sequence listing as an XML file (“Sequence Listing”). The name of the file containing the Sequence Listing is “AF23853.P189WO.xml”. The date of the creation of the Sequence Listing is Sep. 21, 2023. The size of the Sequence Listing is 4,000 bytes. Applicant hereby incorporates by reference the material in the Sequence Listing.

### BACKGROUND

A need exists for the development of methods and systems for efficiently delivering proteins to target cells. Numerous embodiments of the present disclosure aim to address the aforementioned need.

### SUMMARY

In some embodiments, the present disclosure pertains to modified cells. In some embodiments, the modified cells include a fusion protein. The fusion protein generally includes NPC intracellular cholesterol transporter 2 protein (NPC2) and another protein.

In some embodiments, the modified cells include immune cells. In some embodiments, the modified cells include chimeric antigen receptor (CAR) cells. In some embodiments, the CAR cells target an antigen on target cells.

Various proteins may be fused to the fusion proteins of the present disclosure. For instance, in some embodiments, the protein includes, without limitation, non-cytotoxic proteins, therapeutic proteins, fluorescent proteins, enzymes, proteases, RNA-degrading enzymes, toxins, peptides, cyclic dinucleotide synthase, antibodies, nanobodies, single-chain antibodies, or combinations thereof. In some embodiments, the protein is a non-cytotoxic protein. In some embodiments, the protein is a therapeutic protein. In some embodiments, the therapeutic protein is an anti-cancer protein.

The modified cells of the present disclosure may be suitable for use in various applications. For instance, in some embodiments, the modified cells of the present disclosure may be suitable for use in delivering a protein to a target cell. In some embodiments, the modified cells may be suitable for use in delivering the protein to a target cell of a subject in vivo to treat or prevent a condition in the subject.

Additional embodiments of the present disclosure pertain to methods of delivering a protein to a target cell. Such methods generally include associating the target cell with a modified cell of the present disclosure, which includes a fusion protein of NPC2 and the protein to be delivered. Further embodiments of the present disclosure pertain to methods of treating or preventing a condition in a subject by administering to the subject a modified cell of the present disclosure. Additional embodiments of the present disclosure pertain to methods of making the modified cells of the present disclosure. Such methods generally include introducing a fusion protein of the present disclosure to cells.

### DESCRIPTION OF THE DRAWINGS

FIGS. 1A-1H illustrate that mouse NPC intracellular cholesterol transporter 2 (mNPC2) fusion proteins are localized to granules in mouse T cells. FIG. 1A illustrates a proposed mechanism of sorting proteins with M6P residues to granules. FIG. 1B provides a schematic outlining the putative translocation of NPC2 protein through perforin pores. FIG. 1C shows that NPC2 translocation was quantified for the Jurkat cells incubated with purified hNPC2 and perforin proteins using flow cytometry. The mean fluorescence intensity (MFI) of the samples is listed. The NPC2 was labeled using an anti-NPC2-Alexa-488 antibody before being analyzed with a flow cytometer. The flow plots are representative of three independent repeats. FIG. 1D shows representative confocal images of the Jurkat cells incubated with purified human perforin and NPC2 proteins. The nucleus is stained using Hoechst 33342, and the NPC2 is detected using an anti-NPC2-Alexa-488 antibody. Scale bar, 10 μm. FIG. 1E shows a schematic of genetic constructs. mCherry was used to track the localization of mNPC2, and the linker was used to fuse the mNPC2 to the mCherry. FIG. 1F shows a Western blot of the pmel T cells transduced with mNPC2-mCherry construct. FIG. 1G shows a 3D confocal microscopy to visualize the subcellular localization of mNPC2 fused with mCherry in the granules of pmel T cells. The nucleus was stained with Hoechst 33342 and lysosomes were stained with Lysotracker Deep Red. FIG. 1H shows a Manders' overlap coefficient (MOC) to determine the colocalization of the mNPC2 and the lysosomal marker (t-test). For violin plots, the black dotted lines represent the median, and the gray dotted lines denote quartiles. ****p<0.0001; ***p<0.001; **p<0.01; *p<0.05; ns: not significant.

FIGS. 2A-2D illustrate that mNPC2-mCherry is sorted into secretory granules in T cells. FIG. 2A provides a schematic illustrating the localization of mCherry during stages of the T cell interacting with the tumor cell. FIG. 2B provides a representative image of the kinetics of lysosome trafficking recorded by confocal real-time imaging. For the sake of clarity, the outline of the pmel T cell and MC38/gp100 tumor cell are shown in white and green dotted dash lines, respectively. We analyzed a minimum of 30 events for both pmel-mCherry and pmel-NPC2-mCherry T cells. Time is shown in hh:mm and scale bar is 10 μm. FIG. 2C shows quantitative measurements of the distances between the T cell's lysosomes before and after the conjugation with target cells as indicator of lysosomal polarization toward the synapse. The circles indicate the corresponding value for the pmel-mNPC2-mCherry in panel B. FIG. 2D shows lysosomal clustering of pmel T cells demonstrated based on the variance of the distance between the lysosomes immediately after conjugation with the target cells. For violin plot, the black dotted lines represent the median, and the gray dotted lines denote quartiles. ****p<0.0001; ***p<0.001; **p<0.01; *p<0.05; ns: not significant.

FIGS. 3A-3F illustrate that mNPC2-mCherry is transferred to tumor cells. FIG. 3A provides a schematic of co-culture assay. Upon conjugation of T cells and tumor cells, the content of granules within mNPC2-mCherry transduced tumor cells are released into the IS. Perforin monomers bind to the target cell membrane and oligomerize to form perforin pores. Formation of the pore likely enables delivery of mNPC2-mCherry fusion protein into the cytosol of tumor cells. FIG. 3B provides a flow cytometry plot showing the distribution of the mCherry in transduced T cells. FIG. 3C shows that T cells were co-incubated with tumors for 2 hours and the frequency of single tumor cells positive for mCherry expression were determined by flow cytometry. FIG. 3D shows a percentage of the mCherry in MC38/gp100 tumor cells after co-culture assay plotted as Mean±SEM (n=4; t-test). FIG. 3E shows representative images of MC38/gp100 tumor cells incubated with pmel T cells at 1E:1T ratio at different time points. Green indicates labelled tumor cells and red indicates dead cells. Scale bar, 300 μm. FIG. 3F shows that Pmel T cells transduced with mNPC2-mCherry killed the MC38/gp100 tumor cells at the same rate as mCherry pmel T cells (E:T 1:1). ****p<0.0001; ***p<0.001; **p<0.01; *p<0.05; ns: not significant.

FIGS. 4A-4E show the expression and tracking of proteins targeted to granules within human T cells. FIG. 4A shows the schematic of the constructs. The CD19 CAR was cloned with either hNPC2-mCherry or mCherry separated by the self-cleaving T2A peptide sequence. Both constructs included a C-terminal myc epitope tag. FIG. 4B shows phenotyping of the CAR T cells by flow cytometry. FIG. 4C shows subcellular localization of hNPC2 fused with mCherry (hNPC2-mCherry) or mCherry in the granules of the CAR T cells. The nucleus was stained with Hoechst 33342 and lysosomes were stained with Lysotracker Deep Red. FIG. 4D shows colocalization of the hNPC2-mCherry and the lysosomal marker was quantified by MOC. FIG. 4E shows CAR T cells transduced with hNPC2-mCherry killed the NALM-6 tumor cells at the same rate as mCherry CAR T cells (E:T 1:1). For violin plots, the black dotted lines represent the median, and the gray dotted lines denote quartiles. ****p<0.0001; ***p<0.001; **p<0.01; *p<0.05; ns: not significant.

FIGS. 5A-5F shows that chimeric antigen receptor (CAR) T cells transfer the hNPC2-mCherry to the multiple types of tumor cells upon the formation of an IS. FIG. 5A shows a representative image of the kinetics of lysosome trafficking recorded by confocal real-time imaging for CD8⁺ CAR T cells. For the sake of clarity, the outline of the CAR T cell and NALM-6 tumor cell are shown in white and green dotted dash lines, respectively. Applicant analyzed a minimum of 30 events for both mCherry and hNPC2-mCherry CAR T cells. Time is shown in hh:mm and Scale bar is 10 μm. FIG. 5B shows a schematic of Timelapse Imaging Microscopy In Nanowell Grids (TIMING) assay. FIG. 5C shows micrographs showing an example of hNPC2-mCherry or mCherry CAR T cells interacting with and killing a NALM-6 tumor cell. Time is shown in hh:mm and the scale bar is 25 μm. FIGS. 5D-5E show kinetics of synapse formation and subsequent killing mediated by individual CAR T cells. FIG. 5F shows that the transfer of mCherry to tumor cells was assayed by flow-cytometry after co-incubation of the CAR T cells with the tumor cells for 30 min.

FIGS. 6A-6F show CAR NK cells can facilitate the transfer of the hNPC2-mCherry to the tumor cells. FIG. 6A shows a schematic of the constructs. The CD19 CAR was cloned with either mCherry-NPC2 or mCherry separated by the self-cleaving T2A peptide sequence. Both constructs included a C-terminal myc epitope tag. FIG. 6B shows the expression level of mCherry and hNPC2-mCherry in CAR NK cells evaluated using flow-cytometry. FIG. 6C shows subcellular localization of mCherry and hNPC2-mCherry in the granules of the CAR NK cells. The nucleus was stained with Hoechst 33342 and lysosomes were stained with Lysotracker Deep Red. FIG. 6D shows the colocalization of the hNPC2-mCherry and the lysosomal marker, as quantified by MOC. FIG. 6E shows a representative image of the kinetics of lysosome trafficking recorded by confocal real-time imaging. For the sake of clarity, the outline of the CAR NK cell and NALM-6 tumor cell are shown in white and green dotted dash lines, respectively. 30 events for both mCherry and NPC2-mCherry CAR NK cells. Time is shown in hh:mm and scale bar is 10 μm. FIG. 6F shows that tumor cells were assayed for transferring of hNPC2-mCherry by NK cells through the immunological synapse to the tumor cells after 30 minutes of incubation. For violin plots, the black dotted lines represent the median, and the gray dotted lines denote quartiles. ****p<0.0001; ***p<0.001; **p<0.01; *p<0.05; ns: not significant.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Engineering cellular therapeutics by repurposing biological systems has great potential in many fields, including immunology. For instance, the primary mechanism employed by T cells for the specific transfer of proteins at the immunological synapse is via the lysosomal perforin pathway, which facilitates the transfer of cytotoxic granzymes to the target cells. As such, facilitating the delivery of non-cytotoxic proteins through perforin oligomers could dramatically expand the range of protein cargo that T cells can traffic to their target cells.

For instance, T cells and natural killer (NK) cells are one of evolution's cellular engineering masterpieces endowed with the ability to kill abnormal cells with high specificity. Recognition of mutated or foreign peptides restricted in the context of the appropriate major histocompatibility complex (MHC) molecule on the target cell results in the formation of the immunological synapse.

The ability of immune cells to recognize external stimuli and respond by killing target cells and initiating self-proliferation has given birth to a new class of living drugs that is able to dynamically expand and contract in numbers matched to the disease burden. The engineering of synthetic receptors like chimeric antigen receptors (CAR) expressed on a T-cell cellular chassis has shown tremendous potential in cancer therapy. For instance, CD19 CAR T cells targeting the B-cell-specific antigen CD19 expressed in B-cell leukemia and lymphomas have shown durable responses that can last a decade.

The clinical success of CAR T cells has spurred extensive engineering of every element of the CAR, including the extracellular receptor, spacer, and intracellular domains. Synthetic biology approaches have engineered synthetic receptors that allow the T cells to effectively discriminate antigen density or combinatorial antigens on target cells to maximize efficacy while minimizing toxicity. Similarly, the response of the T cells has also been successfully engineered to provide non-native functions, including the expression of additional receptors or the secretion of accessory anti-tumor proteins.

Despite these advances, the ability to take advantage of synapses to facilitate the transport of non-cytotoxic proteins from T cells to the target cells has remained elusive. Engineered immune cells with the ability to deliver on-demand non-cytotoxic cargo with specificity down to individual cells can advance understanding of fundamental biological processes and expand therapeutic options beyond the direct killing of tumor cells.

There are two challenges in delivering non-cytotoxic passenger proteins at the immunological synapse (IS). First, there is limited understanding of the design rules for targeting proteins to the secretory lysosomes of T cells. The secretion of proteins with no directional specificity can be accomplished by appending an appropriate N-terminal leader peptide sequence. However, the mechanisms for sorting proteins into secretory lysosomes are complex. Consequently, a generalized targeting motif has not been identified.

The second challenge is that upon successful degranulation, the contents of the T cell lysosomes are delivered to the synapse, and the passenger protein must be able to either diffuse through perforin pores at the target cell membrane or be taken up efficiently by endocytosis. Unfortunately, there is an incomplete understanding of the features of passenger proteins (electrostatics, sterics, etc.) that can readily be transported across the target cell membrane in a perforin dependent/independent manner.

In sum, a need exists for the development of methods and systems for efficiently delivering proteins to target cells. Numerous embodiments of the present disclosure aim to address the aforementioned need.

### Modified Cells

In some embodiments, the present disclosure pertains to modified cells. In some embodiments, the modified cells include a fusion protein. The fusion protein generally includes NPC intracellular cholesterol transporter 2 protein (NPC2) and another protein. As set forth in more detail herein, the modified cells of the present disclosure can have numerous embodiments.

### Cells

The modified cells of the present disclosure may include various types of cells. For instance, in some embodiments, the modified cells include, without limitation, immune cells, lymphocytes, T cells, mouse T cells, human T cells, mammalian T cells, chimeric antigen receptor (CAR) cells, chimeric antigen receptor (CAR) T cells, CD8⁺ T cells, CD4⁺ T cells, gamma-delta T cells, natural killer (NK) cells, chimeric antigen receptor (CAR) NK cells, macrophages, chimeric antigen receptor (CAR) macrophages, and combinations thereof.

In some embodiments, the modified cells include immune cells. In some embodiments, the modified cells include chimeric antigen receptor (CAR) cells. In some embodiments, the CAR cells target an antigen on target cells. In some embodiments, the antigen includes a CD19 receptor.

In some embodiments, the modified cells include natural killer (NK) cells. In some embodiments, the modified cells include T cells. In some embodiments, the modified cells include CAR T cells. In some embodiments, the CAR T cells target CD19 receptors on target cells.

### Fusion Proteins

Fusion proteins generally include NPC2 and another protein. In some embodiments, NPC2 and the other protein are fused to one another.

In some embodiments, the NPC2 protein of the fusion protein includes SEQ ID NO: 1. In some embodiments, NPC2 includes a sequence with at least 65% sequence identity to SEQ ID NO: 1. In some embodiments, NPC2 includes a sequence with at least 70% sequence identity to SEQ ID NO: 1. In some embodiments, NPC2 includes a sequence with at least 75% sequence identity to SEQ ID NO: 1. In some embodiments, NPC2 includes a sequence with at least 80% sequence identity to SEQ ID NO: 1. In some embodiments, NPC2 includes a sequence with at least 85% sequence identity to SEQ ID NO: 1. In some embodiments, NPC2 includes a sequence with at least 90% sequence identity to SEQ ID NO: 1. In some embodiments. NPC2 includes a sequence with at least 95% sequence identity to SEQ ID NO: 1. In some embodiments, NPC2 includes a sequence with at least 99% sequence identity to SEQ ID NO: 1.

In some embodiments, the NPC2 protein of the fusion protein includes SEQ ID NO: 2. In some embodiments, NPC2 includes a sequence with at least 65% sequence identity to SEQ ID NO: 2. In some embodiments, NPC2 includes a sequence with at least 70% sequence identity to SEQ ID NO: 2. In some embodiments. NPC2 includes a sequence with at least 75% sequence identity to SEQ ID NO: 2. In some embodiments, NPC2 includes a sequence with at least 80% sequence identity to SEQ ID NO: 2. In some embodiments, NPC2 includes a sequence with at least 85% sequence identity to SEQ ID NO: 2. In some embodiments. NPC2 includes a sequence with at least 90% sequence identity to SEQ ID NO: 2. In some embodiments, NPC2 includes a sequence with at least 95% sequence identity to SEQ ID NO: 2. In some embodiments. NPC2 includes a sequence with at least 99% sequence identity to SEQ ID NO: 2.

Various proteins may be fused to the fusion proteins of the present disclosure. For instance, in some embodiments, the protein includes, without limitation, non-cytotoxic proteins, therapeutic proteins, fluorescent proteins, enzymes, proteases, RNA-degrading enzymes, toxins, peptides, cyclic dinucleotide synthase, antibodies, nanobodies, single-chain antibodies, or combinations thereof. In some embodiments, the protein is a non-cytotoxic protein. In some embodiments, the protein is a therapeutic protein. In some embodiments, the therapeutic protein is an anti-cancer protein.

NPC2 may be fused to another protein in various manners. For instance, in some embodiments, NPC2 may be directly fused to the protein. In some embodiments, NPC2 may be fused to the protein through a peptide linker. In some embodiments, the peptide linker includes, without limitation, a flexible linker, a rigid linker, an in vivo cleavable linker, or combinations thereof.

In some embodiments, the peptide linker includes a flexible linker. In some embodiments, the flexible linker includes small, non-polar amino acids (e.g., Glycine) and/or polar amino acids (e.g., Serine and/or Threonine).

In some embodiments, the peptide linker includes a rigid linker. In some embodiments, the rigid linker generally includes α-helical structures. In some embodiments, the rigid linker includes a proline-rich linker.

In some embodiments, the peptide linker includes an in vivo cleavable linker. In some embodiments, the in vivo cleavable linker is cleavable in vivo. In some embodiments, the in vivo cleavable linker is cleavable in response to acidic environments. In some embodiments, the in vivo cleavable linker is an enzyme-cleavable linker. In some embodiments, the in vivo cleavable linker includes one or more disulfide bonds.

In some embodiments, the peptide linker includes SEQ ID NO: 3. In some embodiments, the linker includes a sequence with at least 65% sequence identity to SEQ ID NO: 3.

Fusion proteins may be associated with modified cells in various manners. For instance, in some embodiments, the modified cells of the present disclosure contain the fusion protein. In some embodiments, the fusion protein is within lysosomes of the modified cells. In some embodiments, the fusion protein is within secretory granules of the modified cells.

Fusion proteins may be introduced into modified cells in various manners. For instance, in some embodiments, the fusion protein is transfected into the modified cells.

In some embodiments, the modified cells include a nucleotide that expresses the fusion protein. In some embodiments, the nucleotide is in the form of DNA, RNA, messenger RNA (mRNA), or combinations thereof.

In some embodiments, the introduced nucleotide is in the form of DNA. In some embodiments, the nucleotide is an exogenous gene that expresses the fusion protein. In some embodiments, the exogenous gene is contained in an expression vector, such as a plasmid.

In some embodiments, the nucleotide is in the form of RNA. In some embodiments, the RNA includes a transcript of the fusion protein. In some embodiments, the transcript of the fusion protein is in the form of an mRNA.

The nucleotide expressing a fusion protein may be introduced into cells in various manners. For instance, in some embodiments, the nucleotide is transfected into cells. In some embodiments, the nucleotide is transduced into cells. In some embodiments, the nucleotide is electroporated into cells. In some embodiments, the nucleotide is directly introduced into cells using appropriate delivery agents.

In some embodiments, a gene that expresses the fusion protein of the present disclosure in a modified cell is an endogenous gene. In some embodiments, the endogenous gene represents an edited version of a gene.

### Applications

The modified cells of the present disclosure may be suitable for use in various applications. For instance, in some embodiments, the modified cells of the present disclosure may be suitable for use in delivering a protein to a target cell. In some embodiments, the modified cells may be suitable for use in delivering the protein to a target cell of a subject in vivo to treat or prevent a condition in the subject.

### Delivery of Proteins to Target Cells

Additional embodiments of the present disclosure pertain to methods of delivering a protein to a target cell. Such methods generally include associating the target cell with a modified cell of the present disclosure, which includes a fusion protein of NPC2 and the protein to be delivered. Suitable modified cells, fusion proteins, NPC2 proteins, and proteins to be delivered were described supra and are incorporated herein by reference.

The modified cells of the present disclosure may become associated with target cells in various manners. For instance, in some embodiments, the associating includes incubating the target cells with the modified cells. In some embodiments, the associating occurs in vitro. In some embodiments, the associating occurs in vivo in a subject. In some embodiments, the associating occurs by administering the modified cell to the subject. In some embodiments, the modified cells of the present disclosure are capable of binding to a target cell.

The modified cells of the present disclosure may become associated with various target cells. For instance, in some embodiments, the target cell is a tumor cell. In some embodiments, the tumor cell includes a solid tumor cell. In some embodiments, the solid tumor cells include, without limitation, melanoma cells, ovarian cancer cells, breast cancer cells, glioblastoma cells, lung cancer cells, or combinations thereof. In some embodiments, the target cell is a cancerous cell. In some embodiments, the target cell is a B-cell. In some embodiments, the B-cell is a CD19 expressing B-cell. In some embodiments, the target cell includes solid tumor cells. In some embodiments the target cell is a pathogen infected cell.

Without being bound by theory, proteins may be delivered to target cells through various mechanisms. For instance, in some embodiments, the NPC2 of the fusion protein facilitates the transfer of the protein to the target cell through the lysosomal perforin pathway. In particular embodiments, the NPC2 of the fusion protein facilitates the transfer of the fusion protein to the target cell through perforin pores at an immunological synapse between the modified cell and the target cell.

### Methods of Treating or Preventing a Condition in a Subject

Further embodiments of the present disclosure pertain to methods of treating or preventing a condition in a subject by administering to the subject a modified cell of the present disclosure. As set forth supra, the modified cells of the present disclosure include a fusion protein with NPC2 a protein to be delivered to a target cell. In some embodiments, the protein has a therapeutic effect after being delivered to a target cell. Suitable modified cells, fusion proteins, NPC2 proteins, and proteins to be delivered were described supra and are incorporated herein by reference.

Various methods may be utilized to administer the modified cells of the present disclosure to a subject. For instance, in some embodiments, the administering occurs by methods that include, without limitation, intravenous administration, subcutaneous administration, transdermal administration, topical administration, intraarterial administration, intrathecal administration, intracranial administration, intraperitoneal administration, intraspinal administration, intranasal administration, intraocular administration, oral administration, intratumor administration, local administration, and combinations thereof.

In some embodiments, the administering includes local administration to a specific tissue of the subject. In some embodiments, the tissue includes a tumor.

The methods of the present disclosure may be utilized to treat or prevent various conditions in a subject. For instance, in some embodiments, the condition is cancer. In some embodiments, the cancer includes at least one of leukemia, lymphomas, breast cancer, colon cancer, melanomas, prostate cancer, lung cancer, sarcomas, ovarian cancer, glioblastoma, or combinations thereof.

Without being bound by theory, the methods of the present disclosure may be utilized to treat or prevent conditions in a subject through various mechanisms of action. For instance, in some embodiments, the administered modified cell travels to a specific site of a subject, interacts with the target cell, and delivers the protein to the target cell. In some embodiments, the NPC2 of the fusion protein facilitates the transfer of the protein to the target cell through the lysosomal perforin pathway. In particular embodiments, the NPC2 of the fusion protein facilitates the transfer of the fusion protein to the target cell through perforin pores at an immunological synapse between the modified cell and the target cell.

### Methods of Making Modified Cells

Additional embodiments of the present disclosure pertain to methods of making the modified cells of the present disclosure. Such methods generally include introducing a fusion protein of the present disclosure to cells, where the fusion protein includes NPC2 and another protein.

Various methods may be utilized to introduce fusion proteins into cells. For instance, in some embodiments, the introducing includes transfecting the fusion protein into the cells.

In some embodiments, the introducing includes introducing a nucleotide that expresses the fusion protein into the cells. In some embodiments, the introduced nucleotide is in the form of DNA. RNA, messenger RNA (mRNA), or combinations thereof.

In some embodiments, the introduced nucleotide is in the form of DNA. In some embodiments, the introduced nucleotide is in the form of an exogenous gene. In some embodiments, the exogenous gene is contained in an expression vector, such as a plasmid.

In some embodiments, the introduced nucleotide is in the form of RNA. In some embodiments, the RNA includes a transcript of the fusion protein. In some embodiments, the transcript of the fusion protein is in the form of an mRNA.

Nucleotides that express the fusion proteins of the present disclosure may be introduced into cells in various manners. For instance, in some embodiments, the nucleotide is electroporated into cells. In some embodiments, the nucleotide is transfected into cells. In some embodiments, the nucleotide is transduced into cells. In some embodiments, the nucleotide is directly introduced into cells using appropriate delivery agents.

In some embodiments, the nucleotide is introduced as part of a gene editing system. In some embodiments, the gene editing system edits an endogenous gene form a gene that expresses a fusion protein of the present disclosure.

In some embodiments, a fusion protein or a nucleotide that expresses the fusion protein is introduced into cells in vitro. In some embodiments, a fusion protein or a nucleotide that expresses the fusion protein is introduced into cells in vivo in a subject.

In some embodiments, the methods of the present disclosure also include a step of harvesting cells prior to introducing a fusion protein or a nucleotide that expresses the fusion protein into the cells. In some embodiments, cells are harvested from a subject. In some embodiments, the modified cells are then administered to the subject for treating or preventing a condition in the subject in accordance with the methods of the present disclosure.

### Applications and Advantages

The modified cells and methods of the present disclosure can have numerous applications and advantages. For instance, in some embodiments, the modified cells and methods of the present disclosure may be utilized to facilitate the delivery of non-cytotoxic proteins through perforin pores (i.e., perforin oligomers) at an immunological synapse, which could be utilized to expand the range of protein cargo that modified cells (e.g., T cells) can traffic to numerous target cells.

Unlike the use of cytotoxic proteins like granzyme B, the expression of fusion proteins based on NPC2 (a regulator of amount of cholesterol in T cells) does not adversely affect T cell viability. As such, in some embodiments, T cells expressing NPC2 based fusion proteins can travel to a specific site of disease and interact with the target cells to deliver therapeutic proteins. In some embodiments, the use of NPC2 as a lysosomal delivery protein within T/NK cells will allow for the delivery of exogenous proteins to target cells. In some embodiments, the specificity of cell targeting is dependent on recognition by appropriate T/NK cells.

In some embodiments, NK/T cells expressing NPC2 fusion proteins can be reprogrammed to deliver heterologous non-mammalian proteins at the immunological synapse. In some embodiments, NK/T cells expressing NPC2 fusion proteins can be reprogrammed to deliver recombinant proteins at the immunological synapse.

### Additional Embodiments

Reference will now be made to more specific embodiments of the present disclosure and experimental results that provide support for such embodiments. However, Applicant notes that the disclosure below is for illustrative purposes only and is not intended to limit the scope of the claimed subject matter in any way.

### Example 1. Engineering T/NK Cell Mediated Lysosomal Delivery of Recombinant Proteins at the Synapse (LysoDROPS)

Engineering cellular therapeutics by programming immune (T/NK) cells has great potential in immunology and immunotherapy. The primary mechanism cytotoxic lymphocytes employ for the specific transfer of proteins at the immunological synapse (IS) is via the lysosomal pathway that facilitates the transfer of cytotoxic granzymes, leading to apoptosis in target cells. Facilitating the delivery of non-cytotoxic proteins at the IS will dramatically expand the range of protein cargos that T/NK cells can deliver to the target cells.

In this Example, Applicant demonstrates the engineering of fusion proteins of the intralysosomal protein, NPC2, to mediate the lysosomal mediated delivery of recombinant proteins at the synapse (LysoDROPS). Structural and biophysical considerations suggested that NPC2 could traverse through perforin pores, and in vitro experiments suggested the transport of purified NPC2 through perforin pores on cell membranes. To characterize the ability of NPC2 to facilitate the transfer of payloads, Applicant constructed NPC2-mCherry fusion proteins in T/NK cells. Using confocal microscopy, single-cell imaging, and flow cytometry, Applicant confirmed in mouse TCR CD8⁺ T cells, human CD4⁺ and CD8⁺ chimeric antigen receptor (CAR) T cells, and human CAR NK cells: (a) the colocalization of the NPC2 fused protein with lytic granules, (b) clustering of these lytic granules upon the formation of an IS, and (c) the transfer of the fluorescent protein payload from T cells to target cells in co-culture experiments. These results illustrate that NPC2 can facilitate LysoDROPS to transfer exogenous proteins to target cells upon the formation of an IS.

In particular, Applicant demonstrates in this Example that the small, soluble, native lysosomal protein, NPC intracellular cholesterol transporter 2 (NPC2), can mediate LysoDROPS. By utilizing mCherry as the fluorescent reporter, Applicant demonstrates that NPC2-mCherry fusion proteins are packaged in the lysosome of T cells, are trafficked to the IS as part of secretory lysosomes, and can be detected in the target cells after delivery at the synapse. Applicant also demonstrates that NPC2-mediated delivery is robust and can be accomplished when activated through: (a) the T-cell receptor (TCR) in mouse panel CD8⁺ T cells, (b) CAR (CD19-specific CAR) in both human CD4⁺ and CD8⁺ T cells, and (c) NK cells. The engineered LysoDROPS module can be programmed to transport exogenous proteins to target cells via the IS.

### Example 1.1. Identification of NPC2 as a Putative Lysosomal Chaperone

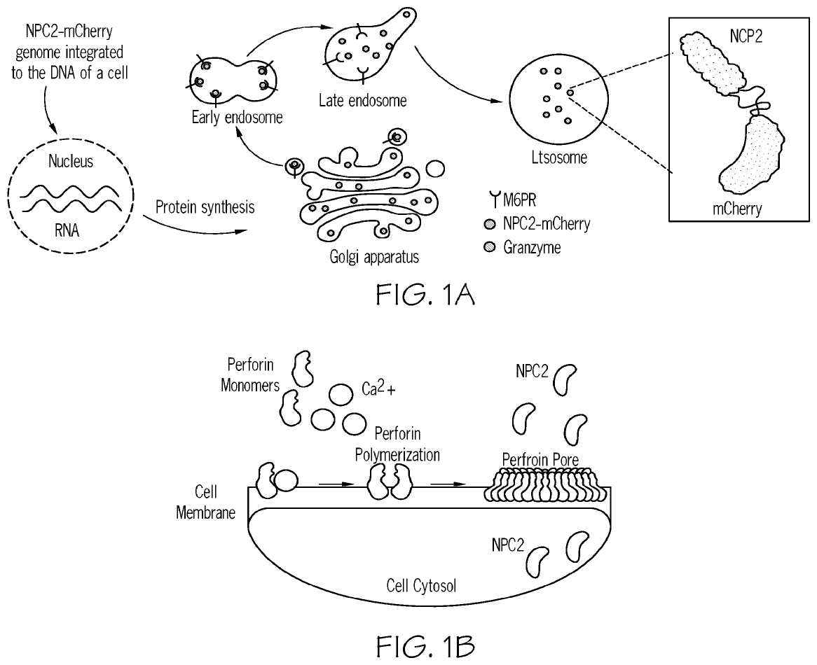

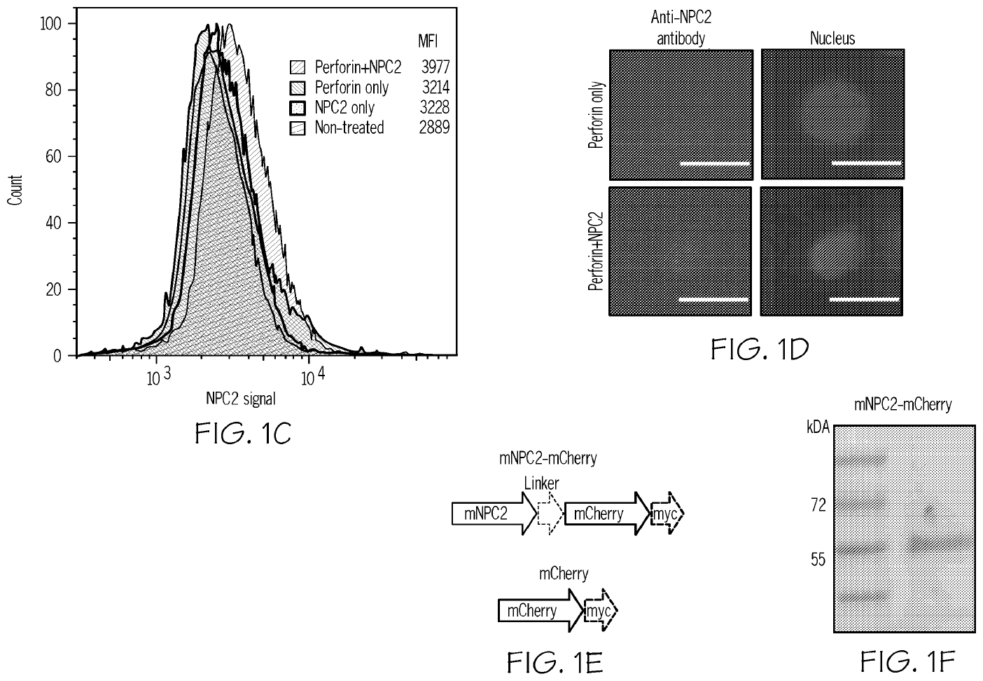

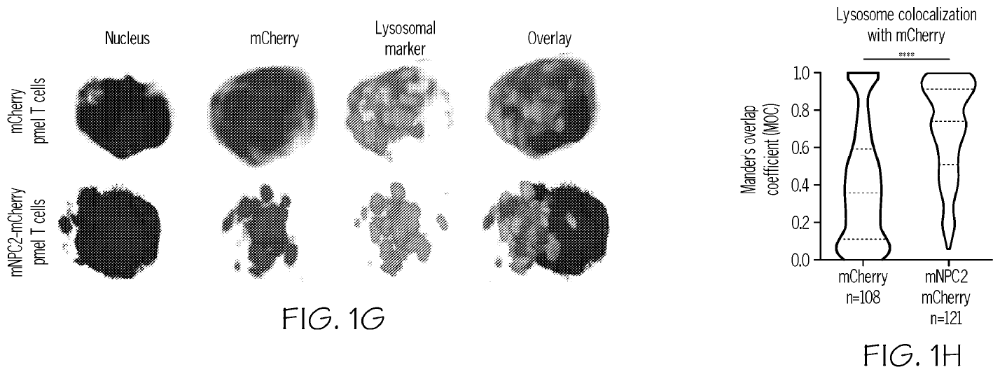

Applicant's first objective was to identify an appropriate partner protein (chaperone) to enable the sorting of recombinant proteins into granules. The majority of lysosomal hydrolases, including granzymes, are modified with mannose 6-phosphate (M6P) residues. Upon recognition of M6P residues, M6P-specific receptors (MPR) bind to the residues to form a clathrin-coated complex that can integrate with early endosomes, and the lysosomal protein gets released into the endosomes (FIG. 1A).

Applicant first investigated the use of granzyme B (GzB) as the lysosomal chaperone, but unfortunately, the expression of GzB was toxic to T cells (not shown), and Applicant focused on the identification of alternate non-cytotoxic proteins. In identifying alternate lysosomal chaperones, Applicant focused on two desirable design considerations: (1) the protein should be able to diffuse through perforin oligomers, and (2) the rate of diffusion through the perforin oligomers should be comparable to GzB.

Applicant chose NPC2 as a soluble candidate lysosomal chaperone protein based on the theoretical considerations associated with diffusion through perforin pores outlined herein. The mechanism of protein translocation through perforin, and specifically the role of electrostatics, is actively debated. Based on the crystal structure of human perforin (from PDB:5KWY), Applicant mapped the electrostatic potential surface and projected this onto the known CryoEM map of oligomerized human perforin. This map suggests that the top of perforin barrel (side closest to the T cell) harbors a positively charged surface, and hence, electrostatics is not a major determinant for the recruitment of passenger molecules to perforin pores. Based on the map, however, the lumen of the perforin barrel is negatively charged and hence can facilitate translocation of positively charged protein cargo. Indeed, the structures of most granzymes present a positively charged electrostatic surface, and the pI of granzymes indicated a net positive charge at neutral pH. Although the pI of NPC2 is 7.6 (positively charged in the lysosomes and likely neutral charge at the synapse), the protein's surface has multiple positively charged amino acids that promote their electrostatic interaction with NPC1 or anionic phospholipids during cholesterol transfer. For these reasons, Applicant hypothesized that, from the perspective of electrostatics, NPC2 translocation through perforin would not be impeded.

The second consideration for the transport across perforin is the delivery rate of the proteins being translocated. Delivery of lysosomal proteins to the target cytosol is a multi-step sequential process, but the translocation across perforin pores is the rate-limiting step (detailed description of the model in the methods section). The rate of delivery of any lysosomal protein by perforin pores can be quantified by the rate constant k_{g} that is inversely proportional to the radius of the molecule, r. Based on the size and structure of NPC2, Applicant predicted the hydrodynamic radius of NPC2 to be ˜2.23 nm, which is smaller than the predicted radius of GzB (˜2.75 nm). When NPC2 is used as a lysosomal chaperone, fusion proteins of sizes <32 kDa are expected to be translocated at a rate comparable to GzB. Thus, from a structural/biophysical perspective, Applicant anticipated that NPC2 should function as a non-toxic lysosomal chaperone with the potential to translocate cargo proteins to the target cytosol.

The expression of proteins in T cells can be impacted by their differentiation status. Using RNA-Seq data (dbGaP: phs002323.v1.p1), Applicant compared the expression of NPC2 across different subsets of CD8⁺ T cells. Unlike GZMB expression that was heavily skewed towards the more differentiated CD8⁺ T cells, NPC2 expression was uniform across all the subsets of CD8⁺ T cells (naïve, central memory, effector memory, and effector). This suggested that the expression of NPC2 was not impacted by the differentiation state of the T cell.

Applicant's next task was to directly test the translocation of the NPC2 protein across perforin pores. Accordingly, Applicant incubated Jurkat cells with purified human perforin (hPerforin) and human NPC2 (hNPC2) proteins and detected the exogenously delivered hNPC2 using both confocal microscopy and flow-cytometry (anti-NPC2 antibody) on fixed Jurkat cells (FIG. 1B). After 2 hours of incubation, cells treated with both hPerforin and hNPC2 showed higher fluorescent intensity compared with cells treated with hNPC2 only (FIG. 1C). This result illustrated that the transport of hNPC2 was at least partially facilitated by hPerforin.

To confirm that the hNPC2 is transported into the cell and is not just present at the target cell membrane, Applicant imaged the target cells using confocal microscopy. Imaging illustrated that the hNPC2 staining was diffuse and localized throughout the cell and not just at the target cell membrane (FIG. 1D). The combined results from both these experiments showed that hNPC2 is internalized into target cells through a mechanism that is at least partially perforin dependent.

Since NPC2 is internalized into the target cell, Applicant next investigated its ability to mediate LysoDROPS to target cells. Applicant evaluated the delivery capacity of NPC2 in three stages: (1) localization to granules in modified T cells, (2) trafficking of NPC2 containing secretory granules to the IS, and (3) delivery and translocation of NPC2 fusion proteins into the target cells.

### Example 1.2. Recombinant NPC2 Fusion Proteins are Sorted into Mouse T Cell Granules

First, Applicant investigated the expression of mouse NPC2-based fusion proteins and their localization in mouse T cells. To track the localization of NPC2 using live-cell microscopy, Applicant constructed a genetic construct In some embodiments, the C-terminus of mouse NPC2 (mNPC2) is fused to mCherry with a flexible hinge linker and the whole construct is fused to a myc epitope tag (FIG. 1E). Applicant also separately cloned the mCherry gene into the same backbone, as a control (FIG. 1E).

Next, Applicant generated retroviral particles and transduced transgenic T cells expressing the anti-gp100 TCR (CD8⁺ pmel T cells). Expression of mNPC2-mCherry had no adverse impact on the health of the pmel T cells, as evident from their normal proliferation rate. Applicant confirmed that full-length mNPC2-mCherry was expressed by western blotting (FIG. 1F).

Applicant stained the transduced pmel T cells using Lysotracker and quantified the localization of mCherry with respect to lysosomal granules (FIG. 1G). As expected by the restricted localization, pmel T cells expressing mNPC2-mCherry showed punctate mCherry staining, whereas pmel T cells expressing mCherry showed diffuse staining throughout the cytoplasm (FIG. 1G). Consistent with this observation, pmel T cells expressing mNPC2-mCherry showed significant localization of mCherry to lysosomal granules (Manders' Overlap Coefficient, MOC, 0.70±0.3) in contrast to pmel T cells expressing mCherry (MOC, 0.40±0.3, p-value<0.0001) (FIG. 1H). This result illustrated that mNPC2 can function as a chaperone and promote the localization of fusion proteins to the lysosome of T cells.

### Example 1.3. MNPC2-mCherry is Localized in Secretory Granules that Traffic to the Immunological Synapse in Mouse T Cells

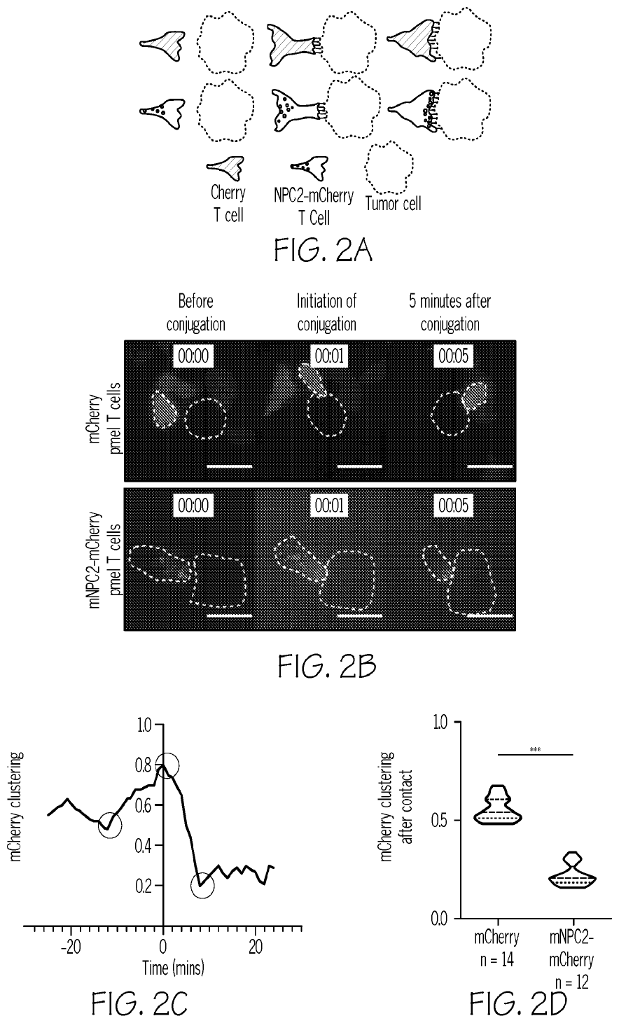

Cytotoxic T lymphocytes (CTLs) mediate the killing of target cells by releasing lytic proteins that are stored in secretory granules at the IS. Dynamic live-cell imaging of the granules can be used to monitor the localization of the secretory granules with respect to the IS (FIG. 2A). Applicant incubated pmel T cells expressing mNPC2-mCherry or mCherry with MC38 target cells presenting gp100 (a tumor-associated antigen) and performed real-time confocal imaging. Within pmel T cells expressing mCherry, the mCherry signal was diffuse both before and after the establishment of the synapse with no evidence of either punctate staining or specific trafficking to the IS (FIG. 2B). By contrast, pmel T cells expressing mNPC2-mCherry showed punctate staining prior to the formation of IS consistent with restricted localization to lysosomal granules (FIG. 2B).

Upon establishment of the IS, the granules were localized to the contact area close to the IS and this localization is consistent with secretory granules that are trafficked to the IS before granule exocytosis permits release of the lysosomal cargo at the IS (FIG. 2B). Applicant quantified the clustering of the granules proximal to the IS by calculating the variance of the distance between the lysosomes. Consistent with clustering, granules within mNPC2-mCherry T cells showed a significantly lower variance (0.23±0.05) in comparison to mCherry T cells (0.56±0.06) (FIGS. 2C-2D). These results illustrate that mNPC2 fusion proteins are localized to secretory granules in T cells that converge at the IS upon recognition of tumor cells.

### Example 1.4. Mouse T Cells Transfer mNPC2 Fused Proteins to the Target Cells

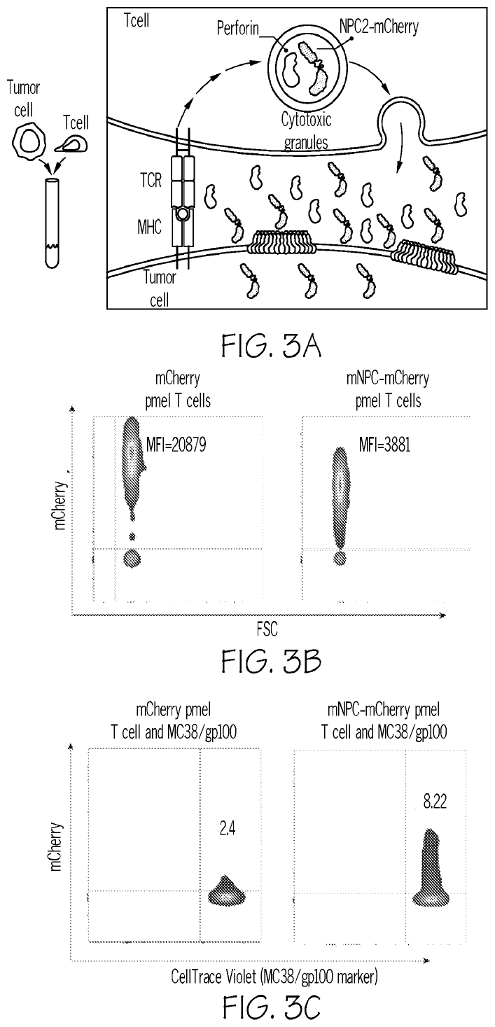

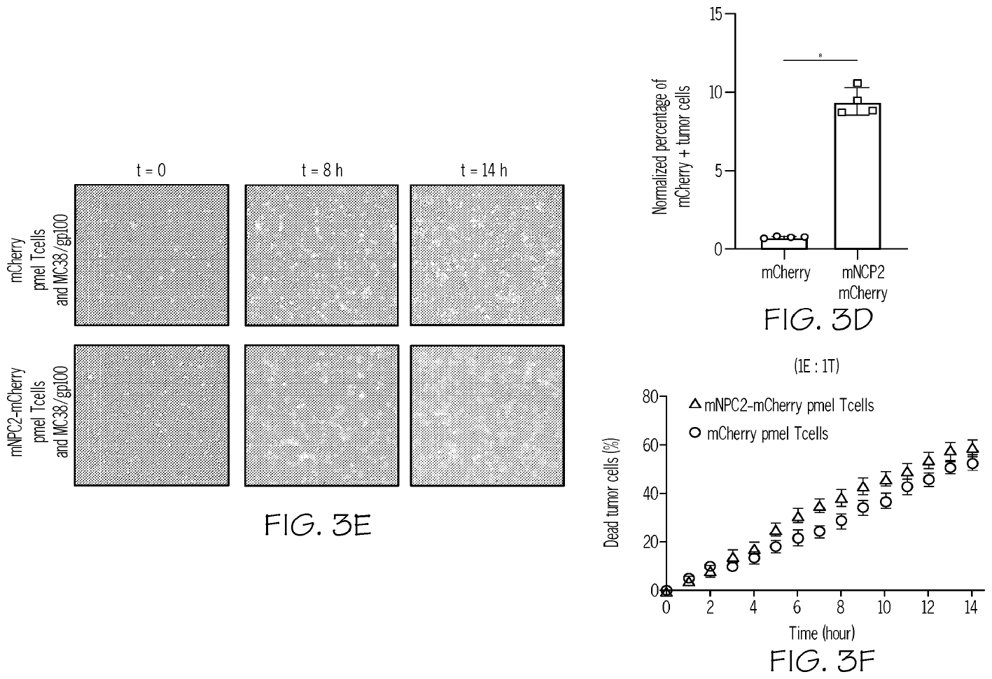

Since the live-cell imaging results suggested that mNPC2-mCherry is localized to the secretory granules in pmel T cells, Applicant aimed to directly quantify mNPC2-mCherry upon successful translocation and delivery into the target cells. Applicant designed co-culture experiments to trace mCherry in target cells to test this hypothesis (FIG. 3A). Accordingly, Applicant incubated labeled MC38/gp100 tumor cells with pmel T cells expressing either mNPC2-mCherry or mCherry for 2 hours and analyzed the cell populations using a flow cytometer. Applicant gated separately live single cells corresponding to both the tumor cells and the pmel T cells and measured mCherry fluorescence in these cells. Pmel T cells expressing mCherry exhibited higher fluorescence intensity compared to pmel T cells expressing mNPC2-mCherry (FIG. 3B). Without being bound by theory, this observation was due to the cytoplasmic expression of mCherry in contrast to the lysosome localized expression of mNPC2-mCherry. Quantitative analysis of the mCherry in tumor cells demonstrated that the MC38/gp100 target cells co-cultured with pmel T cells expressing mNPC2-mCherry showed a significant increase in the mCherry signal (10±1%) compared with target cells co-cultured with pmel T cells expressing mCherry (0.74±0.05%) (FIGS. 3C-3D). These results demonstrate that NPC2-mCherry can facilitate the transfer to mCherry to target cells at the IS.

To evaluate whether the mNPC2 overexpression can impact cytotoxicity of the T cells, Applicant measured the number of MC38/gp100 targets cells killed by pmel T cells using dynamic live cell imaging (FIG. 3E). Tracking the tumor cells during 14 hours of co-incubation with pmel T cells revealed a similar killing rate for T cells expressing mNPC2-mCherry (5±2% h⁻¹) and T cells expressing mCherry (4±1% h⁻¹) (FIG. 3F). Collectively, these data suggest that mNPC2 fused proteins can be translocated into the tumor cells at the IS while preserving T-cell mediated killing.

### Example 1.5. hNPC2 Targets mCherry to Secretory Granules in Human T Cells

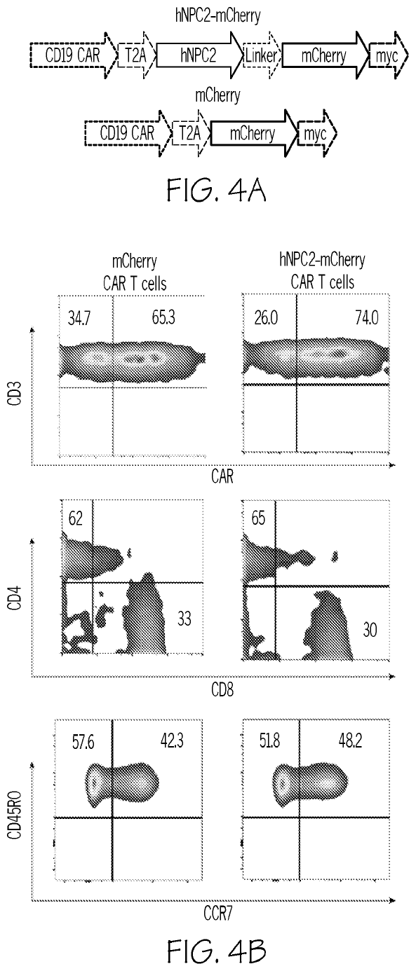

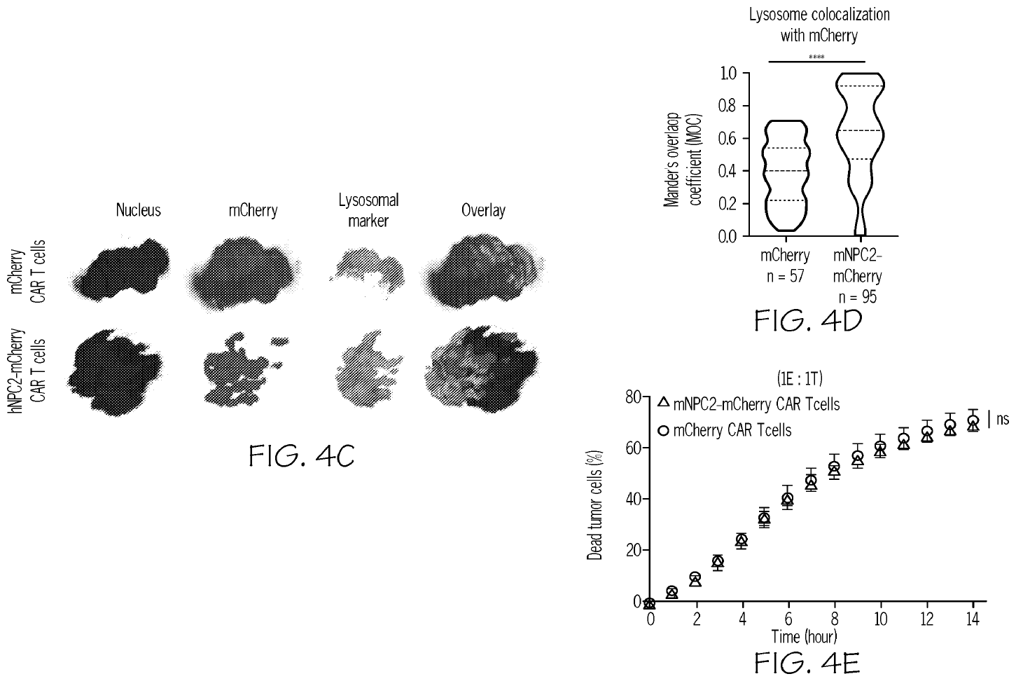

Having established mNPC2 as a molecular delivery chaperone in mouse CD8⁺ T cells, Applicant next aimed to investigate if hNPC2 can function as a lysosomal chaperone in human T cells. Accordingly, Applicant cloned the human NPC2 (hNPC2) downstream of a CD19 chimeric antigen receptor (CAR) gene separated by a self-cleaving peptide, T2A, to enable the expression of both proteins (FIG. 4A). Applicant separately cloned a construct containing the CAR gene followed by mCherry (no NPC2) as a control (FIG. 4A).

Applicant generated retroviral particles and manufactured CAR T cells by a standard 10-day expansion protocol. Phenotyping the cells showed that the majority of T cells expressing both CAR constructs were memory T cells (CD45RO⁺, FIG. 4B). Like the mouse T cell data, human CAR T cells expressing hNPC2-mCherry showed punctate distribution with significant localization to the lysosomal granules (MOC, 0.6±0.3) compared to human CAR T cells expressing mCherry (MOC, 0.4±0.2, p-value<0.0001) (FIGS. 4C-D).

Since the function of NPC2 is to promote cholesterol egress from lysosomes, Applicant next investigated if overexpression of NPC2 would impact the distribution and accumulation of cholesterol within the transduced CAR T cells. To measure the level of cholesterol on the membrane and within the lysosomes, Applicant fixed and permeabilized the transduced CAR T cells and labelled them with Filipin III and visualized the cells by fluorescent microscopy in the UV channel. CAR T cells expressing hNPC2-mCherry showed similar levels of Filipin III staining (MFI, 150±50) compared to mCherry CAR T cells (MFI, 140±70).

Next, Applicant used fluorescently labeled human low-density lipoprotein (LDL) to trace cellular cholesterol deposition within the lysosomes. Applicant incubated the CAR T cells with the BODIPY-FL-LDL for 3 hours and performed confocal imaging of the cells. Both mCherry (800±400) and hNPC2-mCherry CAR T cells (900±400) showed similar levels of cholesterol accumulation within the lysosomes. Overall, these results demonstrate that overexpression of NPC2 does not affect the cholesterol levels within the CAR T cells.

Applicant also compared the cytotoxicity of the CAR T cells against the NALM-6 tumor cells with dynamic live-cell imaging, and confirmed a similar rate of killing for both hNPC2-mCherry (6±2% h⁻¹) and control CAR T cell (6±1% h⁻¹) groups (FIG. 4E).

### Example 1.6. CAR T Cells Efficiently Deliver the hNPC2-mCherry Fusion Protein to Multiple Types of Tumor Cells

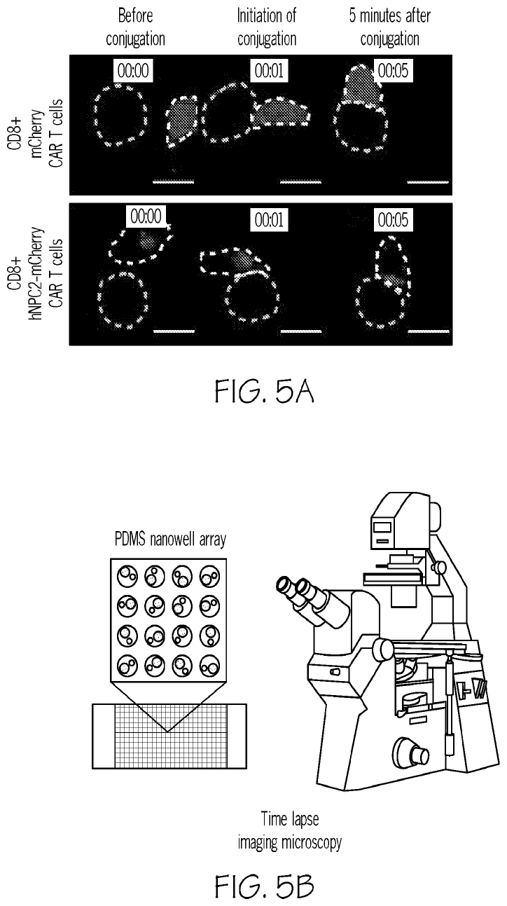

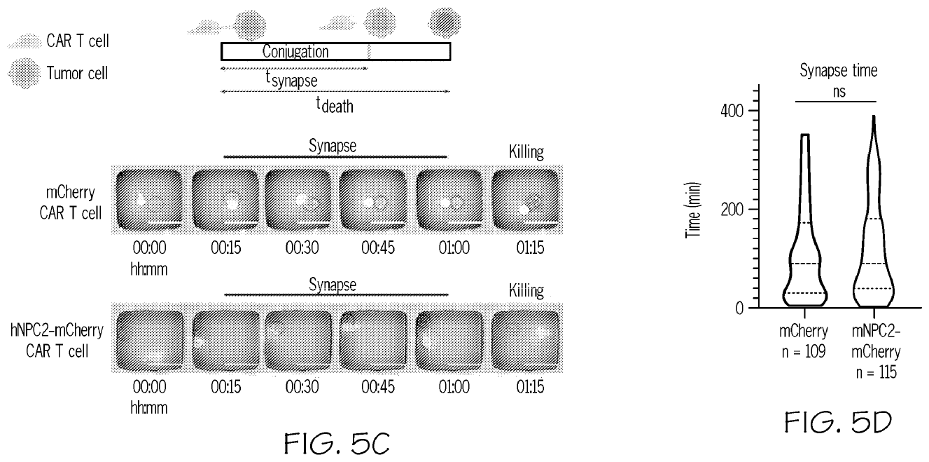

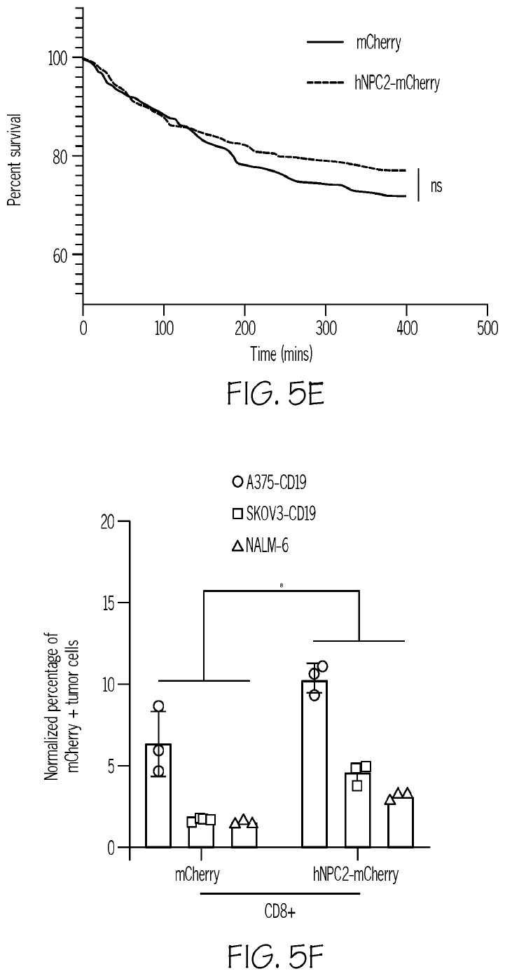

Applicant next used live-cell imaging to evaluate the trafficking of mCherry upon the interaction of the human CAR T cells with CD19 expressing tumor cells, NALM-6. To quantify differences in phenotypes (CD4⁺ and CD8⁺ T cells), Applicant evaluated each of these subsets separately within hNPC2-mCherry and mCherry expressing CAR T cells. Consistent with localization to secretory granules, both CD4⁺ and CD8⁺ T cells expressing hNPC2-mCherry showed punctate staining before the formation of the IS. Similar to the mouse CD8⁺ T cells, upon formation of the IS, both human CD4⁺ and CD8⁺ T cells showed clustered and localized expression of NPC2-mCherry at the contact area close to the synapse (FIG. 5A). These results confirmed that NPC2 functions as a lysosomal chaperone to target mCherry to secretory granules in both CD4⁺ and CD8⁺ human T cells.

Applicant's next objective was to track the killing efficiency of T cells at the single-cell level. Since killing in population level assays can arise from multiple effector cells functioning together to kill tumor cells, Applicant quantified the dynamics of killing at the single-cell ensuring the interaction between individual T cells and tumor cells using Applicant's high-throughput timelapse imaging microscopy in nanowell grids (TIMING) assay (FIGS. 5B-5C). For both NPC2-mCherry CAR T cells that conjugated with the NALM-6 tumor cells and mCherry CAR T cells that conjugated to NALM-6 tumor cells, the duration of synapse was identical (100±100 minutes) (FIG. 5D). Similarly, both types of CAR T cells showed both similar rates and magnitude of killing of NALM-6 tumor cells (FIG. 5E). Collectively, these results demonstrate that overexpression of NPC2 did not impact the quality or quantity of killing mediated by T cells.

Applicant next designed a co-culture experiment of CAR T cells with different types of tumor cells to evaluate the transfer of mCherry from T cells to tumor cells. Applicant utilized three sets of tumor cells expressing CD19: A375-CD19, SKOV3-CD19, and NALM-6 tumor cells and co-incubated them with either CD4⁺ or CD8⁺ CAR T cells. Both CD4⁺ and CD8⁺ T cells were able to transfer hNPC2-mCherry to tumor cells (FIG. 5F). CD8⁺ T cells transferred hNPC2-mCherry to tumor cells at significantly higher rates across all three tumor-cell targets; A375-CD19 (10.4±0.7%), SKOV3-CD19 (4.6±0.6%), and NALM-6 (3.2±0.2%); when compared to CAR T cells expressing mCherry alone with transfer rate of A375-CD19 (6±2%), SKOV3-CD19 (1.66±0.09%), and NALM-6 (1.46±0.08%) (FIG. 5F).

To investigate if the higher capacity of CD8⁺ T cells compared to CD4⁺ T cells for transferring NPC2-mCherry to tumor cells was related to the increase quantity of lysosomes, Applicant quantified the lysosomal volume of both CD4 and CD8 T cells using 3D microscopy, Applicant's results confirmed that per T cell, the lysosomal volume of CD8⁺ T cells (6.5±0.2 μm³) was significantly higher than CD4⁺ T cells (2.93±0.06 μm³), suggesting that CD8⁺ T cells can benefit from the larger lysosomal volume. Taken together, these results, combined with Applicant's results from mouse pmel T cell study, illustrate NPC2-mCherry can be efficiently transferred to multiple types of tumor cells by T cells and the delivery of fusion protein is independent from the type of the T cell immunoreceptor.

The transport of the cytotoxic granules to T-cell membrane and subsequent degranulation at the IS requires influx of Ca²⁺. Importantly, granule exocytosis does not happen in the absence of extracellular Ca²⁺ and is blocked by inhibitors of Ca²⁺ influx like EGTA. Inhibition by EGTA thus provided Applicant a tool to investigate if release of perforin and hNPC2-mCherry via granule exocytosis would impede transfer of mCherry to tumor cells. Since Applicant's co-culture experiments demonstrated that the transfer of mCherry was highest to A375-CD19 tumor cells, Applicant performed co-culture experiments using hNPC2-mCherry CAR T cells and A375-CD19 tumor cells to test this hypothesis. Applicant observed a significant decrease in rate of killing for CAR T cells incubated with EGTA. Pre-incubation with EGTA also significantly impeded the transfer of mCherry to tumor cells, confirming that granule exocytosis was an essential step in the transfer of mCherry mediated by hNCP2-mCherry.

### Example 1.7. NK Cells Expressing the hNPC2-mCherry Localize the Fusion Proteins into the Lysosomes and Deliver them to the Target Cells

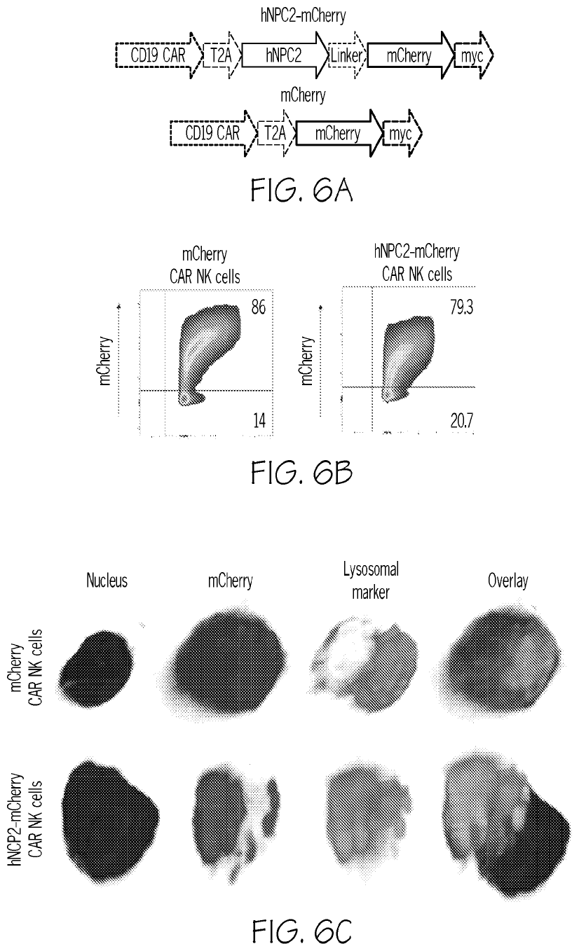

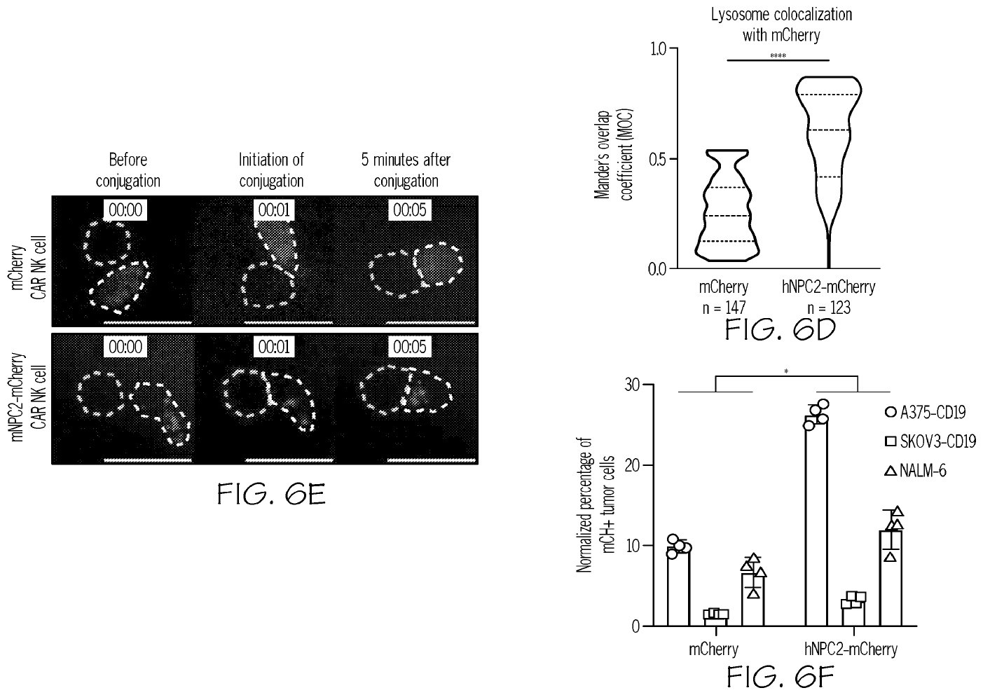

Similar to cytotoxic T cells, killing by NK cells is primarily mediated by granule exocytosis pathway. Therefore, Applicant sought to test the efficacy of translocation for human NPC2 fusion protein from CAR NK cells into the human tumor cells. Applicant used the same constructs and retroviruses as Applicant used for preparation of CAR T cells to transduce the primary NK cells activated with 221mIL21 feeder cells (FIG. 6A). This resulted in a high transduction efficiency of >79% for both mCherry and hNPC2-mCherry constructs (FIG. 6B). Consistent with human T cell data, CAR NK cells expressing hNPC2-mCherry showed punctate distribution with significant localization to the lysosomal granules (MOC, 0.6±0.2) compared to human CAR NK cells expressing mCherry (MOC, 0.3±0.2, p-value<0.0001) (FIGS. 6C-D).

Applicant also observed punctate staining before the formation of the IS and clustered and localized expression of hNPC2-mCherry at the contact area close to the synapse for NK cells expressing hNPC2-mCherry (FIG. 6E). To determine if the hNPC2-mCherry is transferred to tumor cells upon the formation of an IS, Applicant set up the co-culture assay with NK cells and three different tumor cells (A375-CD19, SKOV3-CD19, and NALM-6) as targets. NK cells expressing hNPC2-mCherry transferred significantly more mCherry to A375-CD19 (26±1), SKOV3-CD19 (3.3±0.4), and NALM-6 (12±2) tumor cells compared to NK cells expressing mCherry alone (A375, 10.0±0.7); (SKOV3, 1.53±0.06); (NALM-6, 7±2) (FIG. 6F). These results illustrate NPC2 can be broadly used as a delivery chaperone to transfer mCherry using multiple types of immune cells including T and NK cells.

### Example 1.8. Discussion

T cells have the ability to recognize and respond to even a single copy of their cognate peptide displayed on the target cell, making them very powerful sentinels that patrol the human body. This ability to identify and eliminate single cells with exquisite specificity has enabled their development as living drugs. The engineering and expression of CARs within T cells has expanded the targeting capability of T cells and has accelerated their clinical application. Despite these advances, the cellular response upon activation of the TCR/CAR has primarily focused on cytotoxic responses leading to the death of the target cells. The ability to engineer on-demand LysoDROPS that can transfer exogenous proteins to targets can expand the biotechnological and even clinical application of T and NK cells.

The trafficking of recombinant proteins via the lysosomal pathway is unfortunately not straightforward. The sorting of lysosomal proteins into granules can be either M6P receptor dependent or M6P receptor independent. M6P phosphorylated proteins interact with two different types of receptors: cation-independent and cation-dependent mannose 6-phosphate receptors. Mass spectrometry analysis have revealed multiple randomly distributed M6P sites on soluble lysosomal proteins. These sites are distinguished by their specific conformations that are recognized by multiple enzymes selectively phosphorylating N-linked high mannose oligosaccharides in Golgi compartments. These recognition motifs can be distributed throughout the protein and consequently there is no singular peptide motif that has been identified that can permit the trafficking of recombinant proteins to lysosomes. Although M6P receptors likely play a dominant role in the sorting of lysosomal proteins, at least two alternative receptors have been identified: the lysosomal integral membrane protein (LIMP-2) and sortilin. However, the rules for how sorting is accomplished through these alternate receptors is even less well-characterized. In the absence of defined targeting/trafficking motifs or peptides, the most common well-utilized mechanism to traffic recombinant proteins to the lysosomes is via fusion to native lysosomal proteins. Applicant chose NPC2, a native intralysosomal protein as the chaperone for LysoDROPS in immune cells for several reasons outlined below.

NPC2 displays biophysical characteristics making it an attractive candidate as a lysosomal chaperone. First, NPC2 is a globular protein with a comparatively small size (16 kDa), which can be advantageous for its application in the expression and delivery of fusion proteins. Viral transduction has been shown to be an efficient and safe tool for the expression of proteins in immune cells. Previous studies have revealed the impact of genome size on producer cell mRNA levels, packaging efficiency, and infectivity of the virions. Therefore, NPC2's small size can be beneficial when engineering virions for the delivery of synthetic receptors.

Second, the small size of NPC2 coupled with its globular shape and surface positive charges make it a good candidate to facilitate translocation through perforin pores (16 nm in diameter). The overexpression of NPC2 is also likely to have beneficial effects to T cells. The native function of NPC2 is to facilitate the export of cholesterol out of the lysosomes by enabling the transfer of the cholesterol to the sterol-binding pocket of NPC intracellular cholesterol transporter 1 (NPC1). The NPC2-NPC1 axis thus modulates the total amount of free cholesterol in cells. An increase in the plasma membrane cholesterol level of T/NK cells increases membrane fluidity making the T/NK cells more responsive to receptor signaling and an enhanced ability to form stable IS.

Applicant's work demonstrates the critical first step in engineering of NPC2 as a LysoDROPS chaperone. In designing experiments, Applicant used mCherry as the payload for delivery. Several design considerations will limit the payloads that can be delivered using the NPC2 system including: (1) size, charge, and structure of the protein payload; and its ability to be transported through perforin pores, (2) stability of the payload in the acidic environment of the lysosomes, and (3) the ability of payloads to tolerate fusion at its N-terminus. Fortunately, there are multiple classes of thermostable proteins that satisfy these criteria and hence provide exciting opportunities for the delivery of non-cytotoxic payloads to modify target cells.

Broadly, Applicant has engineered a new module for the delivery of exogenous proteins at the IS via LysoDROPS. Inserting fusion therapeutic proteins into T cell-mediated killing can be a powerful approach to improving the efficacy of engineered T cells by synthetic biology, Applicant proposes that fusion of proteins to NPC2 can be a great tool to deliver proteins that can augment cell death by mechanisms other than granzyme killing. This is particularly important for treatment of non-small cell lung cancer (NSCLC) or melanoma cancer cells that have shown to resist T cell immunity by expressing SERPINB9 protein at higher levels. In this example, delivery of NPC2 fused to more potent tumor cell death inducers can enhance the protection against SERPINB9 expressing tumor cells that are able to inhibit the activity of GzB.

### Example 1.9. Cell Culture

Platinum (plat-E and plat-GP) retroviral packaging cell lines and splenocytes were cultured in DMEM (Gibco cat. #11995065) and MEM α (Gibco cat. #12571089) media, respectively. MC38/gp100 murine colon adenocarcinoma, NALM-6 cells, and PBMCs were cultured in RPMI 1640 medium. All the media were supplemented with 10% (v/v) fetal bovine serum (R&D systems cat. #S11550), 1% (v/v) HEPES (Fisher cat. #15630106), 1% (v/v) Glutamax 100X (Fisher cat. #35-050-061), and 50 mg/ml Normocin (InvivoGen cat. #ant-nr-2). All authenticated cell lines were purchased from ATCC.

### Example 1.10. Construction of Vectors

Murine NPC2 was amplified from Integrated DNA Technologies (IDT) GeneBlock. It has been previously shown that 11 N terminal residues of mCherry are prone to cleavage by the lysosomal proteases. Therefore, Applicant deleted these residues from the construct design. A flexible linker (EFPKPSTPPGSSGGAP-SEQ ID NO: 3) that can span 2.5-2.7 nm was used to create a gap between mNPC2 and mCherry in the fusion protein. Myc-tag sequence was added to the C terminal to detect the proteins' expression by western blotting. All the genes were subcloned into an RVKM vector. The sequences of all constructs were verified. For cloning human NPC2 gene, Applicant prepared cDNA of the U2OS cell line (RRID: CVCL_0042) because of its high expression level of NPC2. Next, Applicant cloned the NPC2, mCherry, and myc genes into a retroviral vector (containing CD19 CAR gene) using NEB HiFi DNA Assembly Master Mix (NEB cat. #E2621S).

### Example 1.11. In Vitro Translocation Assay

Purified human perforin was purchased from Raybiotech (cat. #230-00687). Purified human NPC2 was purchased from Acrobiosystem (cat. #NP2-H52H1). The sublytic concentration of perforin was determined by flow cytometry (15 μg/ml). The Jurkat cells were equilibrated in a solution of 0.4% Bovine serum albumin (BSA), 2 M CaCl₂, and 1% HEPES for 15 minutes before addition of perforin and NPC2 proteins. The cells were incubated with the proteins for two hours and washed twice after the incubation. The cells were washed twice with PBS and fixed with 100 μL IC (intracellular) fixation buffer (eBioscience) for 30 min at RT. Applicant permeabilized the cells for 10 min with 200 μL permeabilization buffer (BD Cytofix solution kit). Applicant performed the intracellular staining using monoclonal rabbit anti-Niemann Pick C2 antibody (abeam cat #218192) overnight at 4° C. Then, Applicant performed secondary antibody staining using Alexa Fluor 488 goat anti-rabbit IgG H&L (abeam cat #150077) for one hour at room temperature. The images of the cells were captured as explained below. The samples were analyzed with a BD LSRFortessa. The flow cytometry data was analyzed with FlowJo software version 10.8 (Tree Star Inc, Ashland, OR, USA).

### Example 1.12. Mathematical Modeling of Translocation Through Perforin Pores

It has been previously shown that a finding the perforin pores by lysosomal proteins is a rate-limiting step and occurs at the rate of k_{g}=3πRₚ²D/l²h², in which Rₚ, l, and h are constant for all proteins and D is the diffusivity of the protein. Applicant can estimate the diffusivity of proteins by the Stokes-Einstein relation, D_{free}=k_{B}T/(6πη_{w}r), where k_{B}T is the thermal energy, η_{w} is the solvent viscosity, and r is the radius of the molecule. Knowing that all the molecules are in the same condition in a solution, Applicant can argue that k_{g} is inversely correlated to the radius of a molecule, r.

### Example 1.13. Retroviral Transduction of Pmel T Cells

Pmel T cells were transduced using the protocol previously described. In brief, Applicant produced retroviral particles by transfecting plat-E cells with the retroviral vectors and packaging plasmids using lipofectamine 2000 transfection reagent (Invitrogen cat. #11668-027). Viral particles were collected after 48 h. Splenocytes were harvested from the spleen of pmel-1 mice and activated with 500 U/ml hIL2 (Proleukin, Bayer Healthcare Pharmaceuticals), 50 mM 2-mercaptoethanol (Fisher cat. #31350010), and 0.5 μg/ml anti-mouse CD3 (BD Bioscience cat. #553057). After 24 h, the viral particles were concentrated using Amicon® Ultra-15 Centrifugal Filter Unit (Millipore cat. #UFC910024). The activated splenocytes were infected by adding the concentrated virus and 1.6 μg/ml Polybrene and spinning the plate at 2000 rpm for 90 minutes. The T cells were propagated for three days and sorted with BD FACS Melody Cell Sorter.

### Example 1.14. Retroviral Transduction of PBMCs and Phenotyping

RD114 expressing PlatGP cells were transfected with the constructs using Lipofectamine LTX Reagent with PLUS Reagent (Thermofisher cat. #A12621). For T cell activation, Applicant coated a non-treated 24-well plate with an anti-CD3 (BD Bioscience cat. #16003785) antibody overnight. The next day, Applicant seeded 1.5×10⁶ PBMCs resuspended in 1 mL of RPMI 1640 media (10% FBS), supplemented with anti-CD28 (Biolegend cat. #302934) antibody, IL7 (Peprotech cat. #200-07), and IL15 (Peprotech cat. #200-15). After 48 hours, the activated T cells were transduced with retroviral supernatants by centrifugation onto a retronectin-coated plate. New media supplemented with IL7 and IL15 was subsequently added to the cells every 2 to 3 days. For phenotyping, CAR T cells were stained for 30 min at 4° C. using a panel of human-specific antibodies CD3 (Biolegend cat. #300328), CD4 (BD Bioscience cat. #563877), CD8 (Biolegend cat. #301006). In addition, cells were stained with the in-house anti-CD19scFv. NK cells were activated by incubating PBMCs with irradiated 221mIL21 feeder cells in presence of IL2 and IL15 cytokines for 10 days. Activated NK cells were centrifuged at low speed (200×G) to remove the dead feeder cells and transduced by the same protocol as human T cells (explained above). The cells were expanded for 10 days before starting the experiments.

### Example 1.15. Confocal Microscopy

For live-cell imaging, transduced T cells were labeled for granules by incubating with LysoTracker Deep Red in media to a final concentration of 100 nM at 37° C. for 1 hour. The nucleus was stained with Hoechst 33342 (Sigma, 14533, 10 μg/ml) for 20 min at 37° C. and washed twice with PBS before acquiring the images. The unlabeled tumor cells and transduced T cells were added to a 96 Well Black Plate glass bottom plate (Thermo Scientific, 160376). 60-70 z-stacks (0.2 μm steps) were captured by a Nikon (Minato, Tokyo, Japan) Eclipse Ti2 inverted microscope equipped with a 100×, Nikon, Plan Apo Lambda, oil, 1.45 NA objective from different fields of view using DAPI, TXRed, and Cy5 channels at 1 min intervals for 1 hour. The composite images were created using NIS-Elements Viewer software. For Filipin III staining, Filipin III (Sigma, SAE0087, 1 mg/ml) was diluted to reach the final concentration of 0.5 mg/ml. Cells were fixed with 4% paraformaldehyde (PFA) and stained for 30 min at 4° C. in dark before acquiring the images. For cholesterol accumulation study, cells were starved for 24 hours for 6 hours and then resuspended in 10 μg/mL BODIPY™ FL LDL (Invitrogen, L34831) for 3 hours before acquiring the images with the confocal microscope.

### Example 1.16. Analysis of Confocal Images

Applicant extracted z-stacks of 16-bit images for each channel and processed them in ImageJ (RRID: SCR_003070) using a series of plugins. First, Applicant applied 3D watershed, 3D objective counter plugin, and 3D ROI Manager plugin to the channels corresponding to mCherry and LysoTracker Deep Red. Then, Applicant obtained the pixel values for each voxel of detected mCherry or Lysosomal marker objects. Next, Applicant used nucleus staining to detect single cells by obtaining their location in the image. Finally, Applicant analyzed all the processed outputs in the R program to calculate the Mender's colocalization coefficient between mCherry and lysosomal marker objects for every single cell. Mander's coefficient (MOC=Σᵢ(Ri×Gi)/√(ΣᵢRᵢ²×ΣᵢGᵢ²)) was used. Applicant quantified the clustering of lysosomes through blob detection using scikit-image library 0.19.2. For each image taken by the confocal microscope, Applicant applied blob detection with Laplacian of Gaussian as kernel to detect the mCherry signal blobs. Applicant only accept detections with a radius between 2-7 pixels and a sigma value of Gaussian higher than 0.03. After detection, Applicant calculated the Euclidean distance between both centroids of each pair of blobs, and their variance is the lysosome clustering value.

### Example 1.17. Co-Culture Experiment

Applicant labeled MC38/gp100 and NALM-6 (RRID: CVCL_0092) cells with Cell-Trace Violet (Invitrogen cat. #C34557) dye at a concentration of 5 μM. The MC38/gp100 cells were loaded into a 6-well plate until they formed their spindle-like morphology. The NALM-6 (RRID: CVCL_0092) cells were seeded in a round-bottom plate. The pmel T cells were incubated with the MC38 cancer cells at a 5:1 effector:target (E:T) ratio. After 2 hours, the media was collected into a round-bottom polystyrene test tube. The cells were washed twice with PBS and resuspended in FACS buffer containing Sytox green (Invitrogen cat. #S34860) at a final concentration of 100 nM to distinguish the live and dead cells. The samples were analyzed with a BD LSRFortessa. The flow cytometry data was analyzed with FlowJo (RRID: SCR_008520). For human T/NK cell study. Applicant incubated the effector cell and target cells at a 1:1 E:T ratio for 30 minutes. For exocytosis inhibition assay, NPC2-mCherry CAR T cells were incubated with pre-labelled NALM-6 tumor cells at E:T ratio of 1:1 in the presence or absence of 5 mM EGTA (MilliporeSigma cat. #324626) for 2 hours.

### Example 1.18. Cytotoxicity Assay

For real-time cytotoxicity assay, Applicant used Cytation 7 Cell Imaging system that allows us to monitor T cell killing over time quickly. CellVue (MilliporeSigma cat. #MINCLARET-1KT) labeled MC38/gp100 cancer cells, and the transduced pmel Tcells were resuspended in MEM media containing Sytox green as death marker. They were incubated in a 96 well-plate at 1:1 effector:target (E:T) ratio under 37° C. and 5% CO2. The images were captured by Cytation 7 inverted microscope using 20× Plan Fluorite WD 6.6 NA 0.45 objective from FITC and TXRed channels at 1 hour timeframe for 24 hours. The primary mask was automatically applied to the images based on the Cy5 channel in Gen5 software. The detected target cells with an intensity unit of higher than 500 were counted as cells killed by pmel T cells. The number of dead cells over time was plotted in Prism software. Similar procedure was followed for human CAR T cells killing assay using NALM6 as target cells.

### Example 1.19. Western Blotting

Transduced pmel T cells (1×10⁶) were lysed in radioimmunoprecipitation assay (RIPA) buffer (2 mM ethylenediaminetetraacetic acid (EDTA), 150 mM NaCl, 50 mM Tris-HCl, and 1% Triton X-100) containing protease inhibitors and phosphatase inhibitors and spun down for 20 min at 13000 rpm at 4° C. The supernatants were quantified for protein concentration using BCA Protein Assay (Pierce, Thermo Fisher Scientific). The protein samples were separated on 4-15% Mini-PROTEAN® TGX™ Precast Protein Gels (Biorad cat. #4561086) and transferred to a Hybond Amersham PVDF transfer membrane (MilliporeSigma cat. #GE10600023 and blocked with 5% skimmed milk in TBST for 1 hour at RT. The PVDF membrane was incubated with the anti-c-myc primary antibody (Biolegend; Clone 9E10) diluted in 2.5% bovine serum albumin (BSA) (1:1000) and kept at 4° C. overnight. The membrane was washed with TBST and then incubated with horseradish peroxidase (HRP)-conjugated secondary antibody at dilution of 1:3000 diluted with 2.5% BSA (Cell Signaling Technology cat. #7076). The protein expression was visualized by using TMB blotting solution.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A method of treating or preventing a condition in a subject, said method comprising:
administering to the subject a modified cell,
wherein the modified cell comprises a fusion protein, wherein the fusion protein comprises NPC intracellular cholesterol transporter 2 protein (NPC2) and a protein to be delivered to a target cell.

2. The method of claim 1, wherein the modified cell travels to a specific site of the condition in the subject, interacts with the target cell, and delivers the fusion protein to the target cell, and wherein the NPC2 of the fusion protein facilitates the transfer of the protein to the target cell through the lysosomal perforin pathway, perforin pores at an immunological synapse between the modified cell and the target cell, or combinations thereof.

3-5. (canceled)

6. The method of claim 1, wherein the administering comprises local administration to a specific tissue of the subject.

7. The method of claim 6, wherein the tissue comprises a tumor.

8. The method of claim 1, wherein the condition is cancer.

9. The method of claim 8, wherein the cancer comprises at least one of leukemia, lymphomas, breast cancer, colon cancer, melanomas, prostate cancer, lung cancer, sarcomas, ovarian cancer, glioblastoma, or combinations thereof.

10. The method of claim 1, wherein the target cell is a B-cell.

11. The method of claim 10, wherein the B-cell is a CD19 expressing B-cell.

12. The method of claim 1, wherein the target cell comprises a tumor cell.

13. The method of claim 1, wherein the modified cell is selected from the group consisting of immune cells, lymphocytes, T cells, mouse T cells, human T cells, mammalian T cells, chimeric antigen receptor (CAR) cells, chimeric antigen receptor (CAR) T cells, CD8⁺ T cells, CD4⁺ T cells, gamma-delta T cells, natural killer (NK) cells, chimeric antigen receptor (CAR) NK cells, macrophages, chimeric antigen receptor (CAR) macrophages, and combinations thereof.

14. The method of claim 1, wherein the modified cell comprises a T cell.

15. The method of claim 1, wherein the modified cell comprises a chimeric antigen receptor (CAR) cell.

16. The method of claim 15, wherein the CAR cell targets an antigen on the target cell.

17. The method of claim 1, wherein the fusion protein is within lysosomes or secretory granules of the modified cell.

18. (canceled)

19. The method of claim 1, wherein NPC2 comprises SEQ ID NO: 1 or a sequence with at least 65% sequence identity to SEQ ID NO: 1.

20. The method of claim 1, wherein NPC2 comprises SEQ ID NO: 2 or a sequence with at least 65% sequence identity to SEQ ID NO: 2.

21. The method of claim 1, wherein the protein is selected from the group consisting of non-cytotoxic proteins, therapeutic proteins, fluorescent proteins, enzymes, proteases, RNA-degrading enzymes, toxins, peptides, cyclic dinucleotide synthase, antibodies, nanobodies, single-chain antibodies, or combinations thereof.

22. The method of claim 1, wherein the protein is a non-cytotoxic protein.

23. The method of claim 1, wherein NPC2 is fused to the protein through a peptide linker, wherein the peptide linker is selected from the group consisting of a flexible linker, a rigid linker, an in vivo cleavable linker, or combinations thereof.

24. (canceled)

25. The method of claim 23, wherein the peptide linker comprises SEQ ID NO: 3 or a sequence with at least 65% sequence identity to SEQ ID NO: 3.

26-81. (canceled)
